(19) United States
(12) Patent Application Publication
(10) Pub. No.: US 2010/0217362 A1
(43) Pub. Date: Aug. 26, 2010

(54) Article of Manufacture & Method for Moisturizing & Relieving Minor Pain

(76) Inventor: Chanda R. Parsons, Decatur, GA (US)

Correspondence Address: Chanda R. Parsons, 4686 Saint James Way, Decatur, GA, 30035, US

(21) Appl. No.: 12/393,453

(22) Filed: Feb. 26, 2009

(51) Int. Cl. A61F 7/00; A41D 19/00

(52) U.S. Cl. 607/111; 607/112; 607/114; 2/163

(57) ABSTRACT

A moisturizing and therapeutic method and article of manufacture by utilizing non-electrical, heat generating objects to provide moisture and temporary pain relief to a user's hand, wherein the non-electrical, heat generating object is held closely against the inside of a user's palm to cause the temperature of the article of manufacture to gradually increase. The article of manufacture has a first glove half formed of natural materials and shaped to cover the back side of a user's hand. A second glove half is formed of the same natural materials to create an inner layer between the first glove half and the third glove layer (sheath). The third glove layer (sheath) is shaped to cover the under side (palm area only) of a user's hand and is joined with the first and second glove halves to form an opening at the wrist for receiving an enveloping a human hand. The article of manufacture has an externally accessible slot or pocket, which is the third glove layer or sheath, in the palm area for receiving and holding non-electrical, heat generating objects.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The present invention relates to a method of moisturizing and temporarily relieving the pain of a user's hand and to an article of manufacture useful in practicing the described method. More particularly, the present invention relates to a method of moisturizing and temporarily relieving the pain of a user's hand by holding a non-electrical heat pack in close proximity to the inside of a user's palm, and an article of manufacture usable for holding the non-electrical heat pack close to the inside of the user's palm.

2. Description of the Prior Art

Moisture heat therapy offers a variety of proven health and beauty benefits. As a favorite to health, wellness and beauty professionals for several years, heat therapy deeply hydrates, softens, soothes, and nourishes dry skin. In addition to aiding in the healing of scars, wounds, and cuts, it also reduces the appearance of crow's feet, fine lines, wrinkles, and lessens the coarseness of the skin.

The overall qualities of warmth and heat have long been associated with comfort and relaxation. Therapists and physicians often treat pain and inflammation with heat therapy by applying it to the body's surface or to deep tissues. The heat increases blood flow and makes connective tissue more flexible. It temporarily decreases joint stiffness, pain, sprains, strains and muscle spasms. Heat therapy also helps reduce inflammation (including various forms of arthritis) and the buildup of fluid in tissues (edema).

Dry heat therapy (i.e. electric heating pads and saunas) draws out moisture from the body and may leave the skin dehydrated. However, moist heat can aid in the heat's penetration into the muscles and some feel that moist heat provides better pain relief. In addition, this method and article of manufacture would be effective for moisturizing and temporary pain relief to the user because of the natural materials being used.

The type of materials used in designing this article of manufacture and method of moisturizing and pain therapy is essential in the effectiveness of the desired results. Utilizing materials that are beneficial to both the user and the environment is a concern that this invention addresses. From the shortage of paper to the depleting ozone layer, the world is trying to discover new ways to become more environmentally conscious. Fortunately, there has been a reasonable solution since the dawn of man; this solution is bamboo. Bamboo and its products have environmentally friendly characteristics and bamboo fabric can be beneficial to people in the following ways: bamboo is a natural fiber that rarely causes allergic reactions to people; it wicks away and manages moisture so that the skin will not become saturated and irritated; it is aggressively anti-bacterial because of a natural property called bamboo kun, which kills up to 70% of any bacteria that tries to incubate; it is breathable and does not cling to the skin regardless of the levels of moisture and perspiration; it sustains temperature extremely well; bamboo molecules absorb energy from the atmosphere and then transfers them into energy for the body, promoting cell activation and good circulation; and bamboo naturally blocks the harmful ultraviolet rays from the sun.

Currently, there are no known products available that combines the treatment of rough, dry, and damaged hands with the treatment of minor aches and pains through non-electrical moisture heat therapy while simultaneously being environmentally conscious with the type of material being used. The present invention provides both a method and an article of manufacture for the dual purposes mentioned above and will be using eco-friendly materials to produce the said article of manufacture.

### BRIEF SUMMARY OF THE INVENTION

The article of manufacture & method for moisturizing and temporarily relieving minor pain of a user's hand via non-electrical heat is designed as a glove that leaves the tips of a user's fingers open to give a more comfortable and natural feel for gripping objects, while keeping the article of manufacture from feeling too cumbersome. Because it is made mostly from natural bamboo fabric, the dual purpose article of manufacture captures and retains moisture from a user's hand while opening the pores for the maximum moisture retention needed to moisturize rough, dry hands. The article of manufacture also uses non-electrical, air activated heat packs in the exterior slots or pockets of each palm to add a more relaxing, therapeutic and rejuvenating experience for any aches or pains present in a user's hand. The inner lining, also comprised of a blend of mostly bamboo and organic cotton fibers, wicks away excessive perspiration and helps the article of manufacture keep the skin ventilated and comfortably dry.

### BRIEF DESCRIPTION OF THE SEVERAL VIEWS OF THE DRAWING

FIG. 1 is an elevation view of the under side or palm area of the article of manufacture.

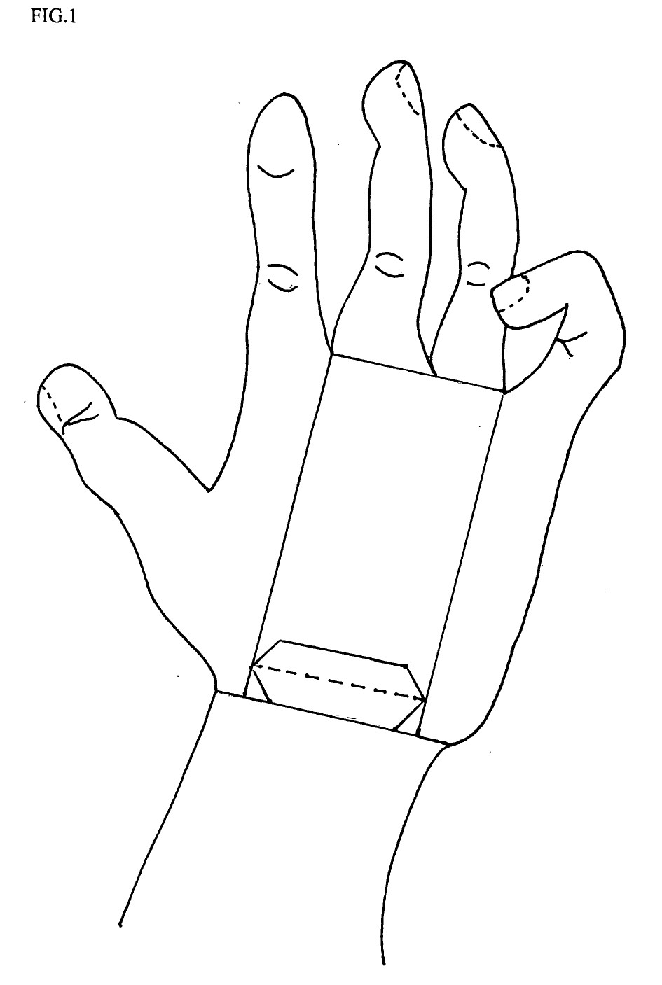

FIG. 2 is a sectional view of the third glove layer (sheath) in which the non-electrical, air activated heat source is placed and held.

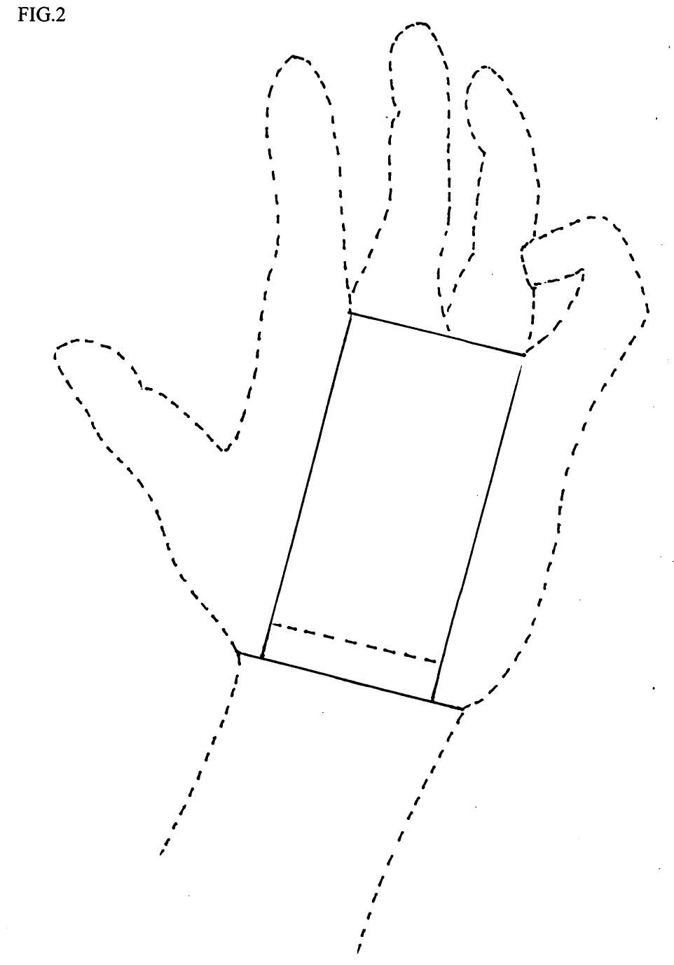

FIG. 3 is an elevation view of the back side of the article of manufacture.

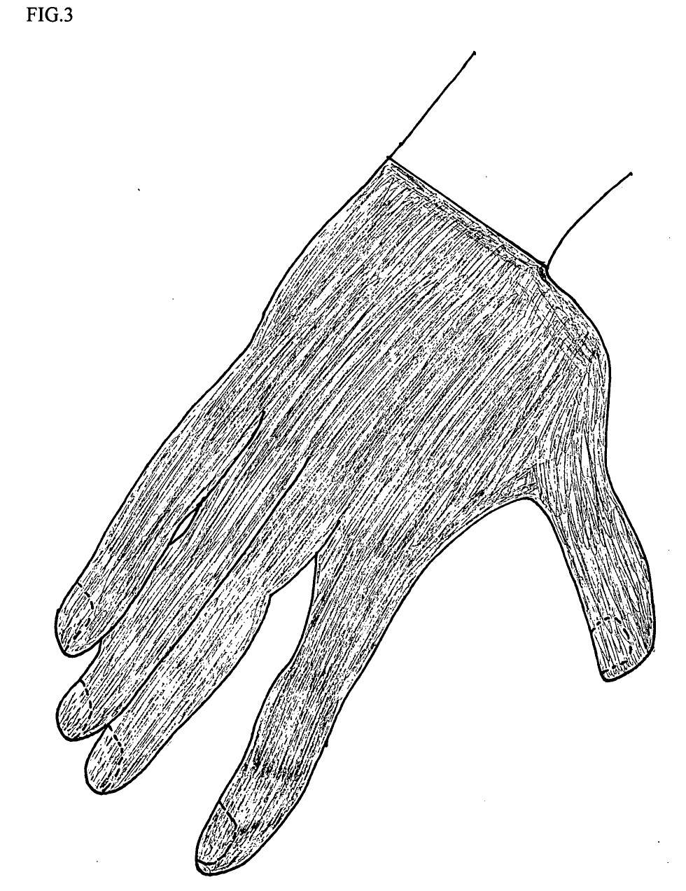

### DETAILED DESCRIPTION OF THE INVENTION

A moisturizing and therapeutic method and article of manufacture for being worn by a user and for storing and holding a non-electrical, air activated heat pack therein to provide moisture and temporary pain relief to a user's hand, via non-electrical, air activated heat; said article of manufacture comprising of an open-finger tip, bamboo & organic cotton blended, glove body comprising of a first glove half which covers the back side of a user's hand and fingers, a second glove half which covers the under side or palm area of a user's hand and fingers, and a third glove layer which covers only the under side or palm area of the user's hand, not the fingers, creating a thin sheath of material that will hold a source or sources of non-electrical, air activated heat close to the user's palm; the first glove half, which covers the back side of a user's hand and fingers, the second glove half, which is the middle layer and covers the under side (palm area) of a user's hand and fingers, is joined with the third glove layer (which is now the exterior layer of the palm area only) to create an opening at the base of the glove. Wherein the wrist area comprises of a stretchable tubular sheath for placement surrounding the entire wrist of a user, extending at least 1″ in length from the base of the palm down towards the wrist area; said wrist sheath begins at the top of a user's wrist having second glove half and third glove layer (sheath) connecting to the base of the user's palm area, and first glove half connecting to the base of the outside portion of the user's hand; the third glove layer (sheath) operatively attached to the second glove half of said article of manufacture and cooperating therewith to form a slot or pocket for receiving and holding said non-electrical heat pack, said palm pocket having at least one air flow port formed as an opening in the center therein, said air flow channel in communication with said pocket, whereby air is able to enter the air flow channel and communicate with the non-electrical air activated heat pack placed in said slot or pocket through the air flow port; wherein said article of manufacture is structured so that when worn by a user and a non-electrical, air activated heat pack is placed into said palm slot or pocket, the air activated heat pack warms the palm and surrounding areas of the user's hand creating moisture while the material comprising the glove retains heat and moisture within the article of manufacture, and air is able to access the heat pack via the one air flow channel.

What is claimed is:

1. A moisturizing and therapeutic article of manufacture for being worn by a user and for storing and holding a non-electrical, air activated heat pack therein to provide moisture and temporary pain relief to a user's hand, via non-electrical, air activated heat; said article of manufacture consists of glove body comprising of a first glove half which covers the back side of a user's hand and fingers, a second glove half which covers the under side or palm area of a user's hand and fingers, and a third glove layer which covers only a portion of the under side or palm area of the user's hand. The third glove layer is operatively attached to the second glove half, creating a thin sheath of material or pocket that will hold a source or sources of non-electrical, air activated heat close to the user's palm; the first glove half, which covers the back side of a user's hand and fingers and the second glove half, which covers the under side (palm area) of a user's hand and fingers, are joined together to create an opening at the base of the glove to receive and hold a user's hand. The second glove half and third glove layer (pocket) are operatively attached creating a second opening at the base of the glove, serving also as an air flow channel, for receiving and holding said non-electrical heat pack, air activated heat pack.

2. (canceled)

3. The moisturizing and therapeutic article of manufacture further comprising that the inner layer of material, which is the base of the slot or pocket (from the palm view), thereby limits the direct thermal interaction of the heat with a user's palm area of the hand.

4. A method of actively moisturizing and providing pain relief to a user's hand, specifically the palm area, said method comprising of said palm pocket or slot having at least one air flow port formed as an opening at the base of the glove body therein, said air flow channel in communication with said pocket and its contents, whereby air is able to continuously enter the air flow channel and communicate with the non-electrical, air activated heat pack placed in said slot or pocket through the air flow port; wherein said article of manufacture is structured so that when worn by a user and a non-electrical, air activated heat pack is placed into said palm slot or pocket, the air activated heat pack warms the palm and surrounding areas of the user's hand to thereby cause a thermal exchange between the heat pack and the inside of the user's palm while material comprising the glove retains moisture and heat within the article of manufacture.
